(19) United States
(12) Patent Application Publication
(10) Pub. No.: US 2025/0084440 A1
(43) Pub. Date: Mar. 13, 2025

(54) BIOLISTIC PARTICLE DELIVERY COMPOSITIONS, METHODS, AND SYSTEMS

(71) Applicant: INARI AGRICULTURE TECHNOLOGY, INC., Cambridge, MA (US)

(72) Inventor: Daniel Bruznican, Cambridge, MA (US)

(21) Appl. No.: 18/727,575

(22) PCT Filed: Dec. 1, 2022
(86) PCT No.: PCT/US2022/080753

§ 371 Date: Jul. 9, 2024

Related U.S. Application Data
(60) Provisional application No. 63/266,609, filed on Jan. 10, 2022.

(51) Int. Cl. C12N 15/89

(52) U.S. Cl. CPC C12N 15/895

(57) ABSTRACT

Macrocarriers with a hydrophobic and/or proteinaceous coating are disclosed. Such coated macrocarriers can increase transfer efficiency of microcarriers into targeted cells when used in a biolistic particle delivery device.

### CROSS-REFERENCE TO RELATED APPLICATIONS

This application is a PCT International Application which claims the benefit of U.S. Provisional Application Ser. No. 63/266,609 filed Jan. 10, 2022, which is incorporated herein by reference in its entirety.

### BACKGROUND

Biolistic particle delivery is a technique by which exogenous DNA, RNA, protein, and/or biological materials are introduced into cells via firing microparticles coated with the biological material into cells by force. This technique, also known as particle bombardment, is a versatile method of genetic modification for plant and animal tissue by introducing desired genetic information into targeted cells.

A biolistic particle delivery system, also known as a “gene gun,” is a device used to introduce exogenous DNA, RNA, protein, and/or biological materials into cells. FIG. 1 shows a diagram of a conventional particle delivery system using a macrocarrier lacking a coating. The left side of the drawing depicts the system before discharge. A pressurized gas, typically helium, fills the gas acceleration tube wherein pressure builds against a rupture disk. The pressure then reaches a point wherein the rupture disk releases, or “breaks,” and the resulting burst of helium propels a macrocarrier loaded with microparticles on the surface of the macrocarrier, into a stopping screen. When the macrocarrier contacts the stopping screen, the microcarriers are propelled through the screen and into the target cells. The right side of FIG. 1 depicts the system immediately after discharge while the microcarriers are still in flight. When the microparticles comprise biological macromolecules, the macromolecules are introduced into the target cells. In an embodiment wherein the biological macromolecules comprise a DNA molecule comprising a gene, introducing the biological macromolecules into a target cell can induce target cell transformation. In an embodiment wherein the biological macromolecules comprise a gene-editing reagent, introducing the biological macromolecules into a target cell can induce gene-editing. A typical biolistic particle delivery system is Biolistic® PDS-100/He manufactured by Bio-Rad (Hercules, CA), catalog numbers 165-2257, 165-2250LEASE to 165-2255LEASE.

There are many advantages of biolistic particle delivery. For example, the gas-accelerated particles can penetrate into deeper or shallower layers of the targeted tissue based on adjustment of the discharge pressure, rupture disk selection, distance from the rupture disk to the macrocarrier, the macrocarrier travel distance to the stopping screen, and/or the distance between the stopping screen and the target cells. Moreover, many different tissues and cell types can be transformed with biolistic particle delivery and the system can be adapted to deliver DNA, RNA, protein, or a combination of biological material. For example, Miller et al. (Nature Scientific Reports 11:7695, 2021) disclose a double-barrel biolistic delivery system and related methods where DNA-coated gold nanoparticles in a 50% ethanol/water mixture were placed in two desired positions on a macrocarrier for simultaneous delivery to target cells. Furthermore, the biolistic delivery protocol is relatively simple and fast. Nonetheless, it would be desirable to increase the rates of biolistic particle-mediated transformation and gene editing in target cells.

### SUMMARY

The present disclosure provides a coated macrocarrier comprising a macrocarrier adapted for use in a biolistic particle delivery system and a coating on at least a portion of a surface of the macrocarrier. In an embodiment, the coating comprises a hydrophobic material. In another embodiment, the coating comprises a proteinaceous material. In an embodiment, the coated macrocarrier further comprises a plurality of microcarriers on a surface of the coated macrocarrier, wherein the microcarriers can be coated with a composition comprising one or more biological macromolecules (DNA, RNA, a polynucleic acid, a protein, a ribonucleoprotein, a gene-editing reagent, (e.g., Cas nucleases and guide RNAs (gRNAs), a ribonucleoprotein comprising a Cas nuclease and a gRNA, transcription activator-like effector nucleases (TALENs), artificial zinc finger nucleases, and/or a donor DNA template).

The present disclosure also provides a method of making a coated macrocarrier adapted for use in a biolistic particle delivery system comprising applying a hydrophobic and/or a proteinaceous coating to at least a portion of a surface of a macrocarrier which lacks the coating, thereby obtaining a coated macrocarrier adapted for use in a particle delivery system. In an embodiment, the coated macrocarrier further comprises a plurality of microcarriers on a surface of the coated macrocarrier, wherein the microcarriers can be coated with a composition comprising one or more biological macromolecules.

The present disclosure further provides a biolistic particle delivery system comprising a biolistic delivery apparatus comprising a gas acceleration tube, a rupture disk, a coated macrocarrier according the disclosure herein, and a stopping screen. In an embodiment, the coated macrocarrier further comprises a plurality of microcarriers on a surface of the coated macrocarrier, wherein the microcarriers can be coated with a composition comprising one or more biological macromolecules. In an embodiment, the biolistic particle delivery system further comprises target cells oriented in the system to receive the microcarriers. The present disclosure further provides a method of introducing one or more biological macromolecules into a cell, wherein the method comprises discharging the biolistic particle delivery system comprising a coated macrocarrier thereby introducing one or more biological macromolecules into a cell. In an embodiment, the introducing of the one or more macromolecules into the target cells with the coated macrocarrier is increased in comparison to a control system with an uncoated macrocarrier, resulting in an increased rate of target cell transformation and/or an increased rate of gene-editing. In another embodiment, the transfer efficiency of microcarriers from the coated macrocarrier is increased in comparison to the transfer efficiency of a control system comprising an uncoated macrocarrier resulting from a reduction in retention of the microcarriers on the coated macrocarrier after the discharging.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 shows a diagram of a conventional particle delivery system using a macrocarrier lacking the hydrophobic coating. The left side of the drawing depicts the system before discharge and the right side of the drawing depicts the system immediately after discharge while the microcarriers are still in flight.

FIG. 2 shows at the top a macrocarrier seated in a holder to which 12 microliters of a solution of 100% ethanol containing gold nanoparticles was just applied to an untreated macrocarrier (left), and spread within a few seconds over the entire surface of the macrocarrier (right); and at the bottom a PTFE-treated macrocarrier seated in a holder with a solution of 100% ethanol containing gold nanoparticles stably localized near the center of the macrocarrier.

FIG. 3 is a photograph of two macrocarriers which were used to deliver gold nanoparticles using a Bio-Rad Biolistics® PDS-1000/He Particle Delivery System. At left in the figure is a PTFE-coated macrocarrier after use and at right is an untreated macrocarrier after use.

FIG. 4 is a closeup photograph showing gold sticking to an untreated macrocarrier after use.

FIG. 5 shows fluorescence images of an immature maize embryo four days after bombardment with the indicated microcarrier amounts. The microcarriers were 0.6 μm gold nanoparticles coated with a TransIT-2020® solution having a fluorescent marker and selection marker-encoding plasmid. RNPs (Cas protein bound with RNA) and a short DNA oligonucleotide donor template for non-homologous end joining (NHEJ) insertion in the maize genome. The top panels show images of maize embryos bombarded with 400 μg of the coated gold nanoparticles utilizing an untreated macrocarrier (top most panel, left) or with 400 μg of the coated gold nanoparticles utilizing a PTFE-treated macrocarrier (top most panel, right). The middle panels show images of maize embryos bombarded with 1200 μg of the coated gold nanoparticles utilizing an untreated macrocarrier (middle panel, left) or with 1200 μg of the coated gold nanoparticles utilizing a PTFE-treated macrocarrier (middle panel, right). The bottom-most panels show images of maize embryos bombarded with 3000 μg of the coated gold nanoparticles utilizing an untreated macrocarrier (bottom panel, left) or with 3000 μg of the coated gold nanoparticles utilizing a PTFE-treated macrocarrier (bottom panel, right).

Various embodiments of the present disclosure will be described in detail with reference to the drawings, wherein like reference numerals represent like parts throughout the several views. Reference to various embodiments does not limit the scope of the disclosure. Figures represented herein are not limitations to the various embodiments according to the disclosure and are presented for illustration of the disclosure.

### DETAILED DESCRIPTION

The present disclosure relates to macrocarriers adapted for use in a biolistic particle delivery system, wherein the macrocarriers comprise a coating on a surface of the macrocarrier. In an embodiment, the coating comprises a hydrophobic coating and/or a proteinaceous coating. In an embodiment, a coated macrocarrier further comprises a plurality of microcarriers on the surface of the macrocarrier having the coating, optionally wherein the microcarriers are nanoparticles comprising gold, tungsten, steel, or an alloy thereof. In an embodiment, the microcarriers are coated with a composition comprising one or more biological macromolecules (DNA, RNA, a polynucleic acid, a protein, a ribonucleoprotein, a gene-editing reagent, (e.g., Cas nucleases and guide RNAs (gRNAs), a ribonucleoprotein comprising a Cas nuclease and a gRNA, TALENs, artificial zinc finger nucleases, and/or a donor DNA template). The present disclosure also relates to a method of making a coated macrocarrier adapted for use in a biolistic particle delivery system wherein the coating is applied to at least one surface of the macrocarrier. In an embodiment, the method further comprises applying a microcarrier composition to the coated surface of the macrocarrier, optionally wherein the microcarrier composition is applied at, or near, about the center of the macrocarrier. The present disclosure also relates to a biolistic particle delivery system comprising a biolistic delivery apparatus comprising a gas acceleration tube, a rupture disk, a coated macrocarrier as disclosed herein, and a stopping screen. In an embodiment, the system further comprises a plurality of microcarriers on the surface of the coated macrocarrier wherein the microcarriers are coated with a composition comprising one or more biological macromolecules. In an embodiment, the system further comprises a target cell oriented in the system to receive the microcarriers coated with the one or more biological macromolecules. The present disclosure also relates to a method of introducing one or more biological macromolecules into a cell, wherein the method comprises discharging the biolistic particle delivery system described herein, introducing one or more biological macromolecules into a cell.

The embodiments described herein are not limited to any particular device or method of using the device, which can vary and are understood by skilled artisans based on the present disclosure herein.

Numeric ranges recited within the specification are inclusive of the numbers within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range (e.g. 1 to 5 includes 1, 1.5, 2, 2.75, 3, 3.80, 4, and 5).

So that the present disclosure can be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation. Further, units, prefixes, and symbols can be denoted in its SI accepted form. In describing and claiming the embodiments of the present disclosure, the following terminology will be used in accordance with the definitions set out below.

The singular forms “a,” “an,” and “the” can include plural referents unless the content clearly indicates otherwise.

The term “about,” as used herein, refers to variations in size, distance or any other types of measurements that can be resulted from inherent heterogeneous nature of the measured objects and imprecise nature of the measurements itself. The term “about” also encompasses variation in the numerical quantity that can occur, for example, through typical measuring or handling procedures in the real world; through inadvertent error in these procedures; through differences in the manufacture, source, or purity of the ingredients used to make the device or carry out the methods, and the like. Whether or not modified by the term “about”, the claims include equivalents to the quantities.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

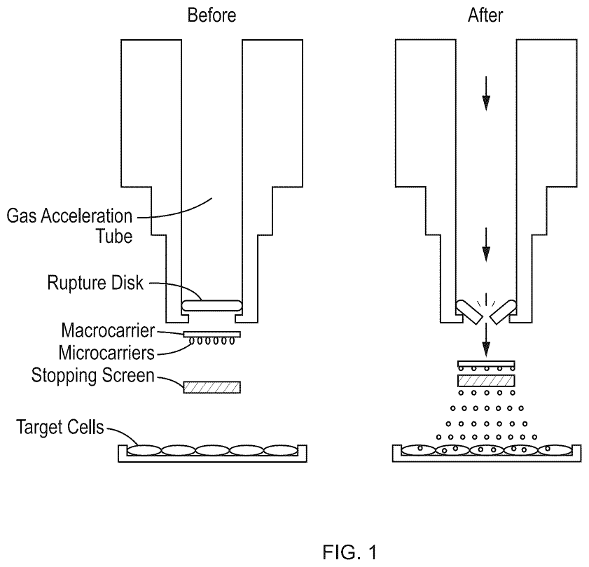

The present disclosure provides a coated macrocarrier. As used herein, “coated macrocarrier” refers to a macrocarrier wherein at least one part of at least one surface of the macrocarrier comprises a coating. In an embodiment, the macrocarrier is adapted for use in a biolistic particle delivery system. A conventional biolistic particle delivery system is shown, generally, in FIG. 1. The macrocarriers are also known as microparticle carrier disks. Useful macrocarriers are sold by Bio-Rad (Hercules, CA), catalog number 165-2335.

Macrocarriers of the present disclosure can be any shape and any size. In an embodiment, the macrocarrier is in the shape of a flat, circular disk. In an embodiment, the macrocarrier is of any suitable shape and size that is adaptable for use in a biolistic particle delivery system. The macrocarriers of the present disclosure can be made of any material. In an embodiment, the macrocarrier comprises a material suitable for use in a biolistic particle delivery system. In an embodiment, the macrocarrier comprises a plastic. In certain embodiments, the plastic can comprise the plastic used in a macrocarrier sold by Bio-Rad (Hercules, CA) under catalog number 165-2335. In certain embodiments, the macrocarrier is the macrocarrier sold by Bio-Rad (Hercules, CA) under catalog number 165-2335.

In an embodiment, the coating comprises a hydrophobic material. In an embodiment, the coating comprises a hydrophobic polymer. In an embodiment, the hydrophobic polymer is a fluoropolymer or a mixture of fluoropolymers. In yet another embodiment, the fluoropolymer comprises PTFE (polytetrafluoroethylene), PVDF (polyvinylidene fluoride), PCTFE (polychlorotrifluoroethylene), a copolymer thereof, or a mixture thereof.

In an embodiment, the coating comprises a proteinaceous material. In an embodiment, the proteinaceous material comprises a protein. In an embodiment, the protein comprises bovine serum albumin.

In an embodiment, at least one portion of at least one surface of the macrocarrier comprises the coating. In an embodiment, the entirety of the surface of the macrocarrier comprises the coating. In another embodiment, at least one side of the macrocarrier comprises the coating. In another embodiment, a portion of one side, and/or a portion of all sides of the macrocarrier comprise the coating. In an embodiment, a center portion of a surface of the macrocarrier comprises the coating. In another embodiment, the macrocarrier comprises more than one coating. In an embodiment, wherein the macrocarrier is a flat disk, one coating is on one flat surface of the macrocarrier and another, distinct coating is on the opposite flat surface. In an embodiment, one surface of the macrocarrier comprises more than one coating.

In an embodiment, the coated macrocarrier further comprises a plurality of microcarriers on the surface of the macrocarrier having the coating.

As used herein, “microcarriers” are particles as used in a biolistic particle delivery system. Microcarriers can have any shape. In an embodiment, the microcarriers are at least roughly or essentially spherical. In an embodiment, the microcarriers are inert nanoparticles. In an embodiment, the microcarriers are nanoparticles with a diameter of about 0.1 to about 2 μm. In an embodiment, the microcarriers comprise gold, tungsten, steel, palladium, rhodium, platinum, iridium, another heavy metal, or an alloy thereof, or a combination thereof.

In an embodiment, the microcarriers are coated with a composition comprising one or more biological macromolecules. In an embodiment, the biological macromolecules comprise DNA, RNA, a polynucleic acid, a protein, a ribonucleoprotein, genetic material, a gene-editing reagent, (e.g., Cas nucleases and guide RNAs (gRNAs), a ribonucleoprotein comprising a Cas nuclease and a gRNA, TALENs, artificial zinc finger nucleases, and/or a donor DNA template). or combination thereof.

The present disclosure provides a method for making a coated macrocarrier as described herein. The macrocarrier can be adapted for use in a biolistic particle delivery system. Such a method comprises applying a coating to at least a portion of one surface of a macrocarrier thereby obtaining a coated macrocarrier.

In an embodiment, the method comprises applying a coating comprising a hydrophobic material. In an embodiment, the method comprises applying a coating comprising a hydrophobic polymer. In an embodiment, the hydrophobic polymer is a fluoropolymer or a mixture of fluoropolymers. In an embodiment, the fluoropolymer comprises PTFE (polytetrafluoroethylene), PVDF (polyvinylidene fluoride), PCTFE (polychlorotrifluoroethylene), a copolymer thereof, or a combination thereof. In an embodiment, the coating comprises PTFE.

In an embodiment, the method comprises applying a coating comprising a proteinaceous material. In an embodiment, the proteinaceous material comprises one or more protein(s). In an embodiment, the protein comprises bovine serum albumin.

In an embodiment, the method comprises applying a coating to at least one portion of at least one surface of the macrocarrier. In an embodiment, the method comprises applying a coating to the entirety of the surface of the macrocarrier. In an embodiment, wherein the macrocarrier is a flat disk, the method comprises applying a coating on one flat surface of the macrocarrier. In another embodiment, the method comprises applying a coating to at least one side of a macrocarrier. In another embodiment, the method comprises applying a coating to a portion of one side, and/or a portion of all sides of a macrocarrier. In an embodiment, the method comprises applying a coating to a center portion of a surface of a macrocarrier. In another embodiment, a coating is applied in two desired positions on a macrocarrier (e.g., in alignment with each barrel of a double-barrel biolistic delivery system (Smith et al. Nature Scientific Reports 11:7695, 2021)). In another embodiment, the method comprises applying more than one coating to a macrocarrier. In an embodiment, wherein the macrocarrier is a flat disk, the method comprising applying a coating on one flat surface of the macrocarrier and applying another coating on the opposite flat surface. In an embodiment, the method comprises applying more than one coating onto a surface of the macrocarrier. In another embodiment, the method comprises applying a coating to at least the center portion of at least one surface of a macrocarrier.

In the method described herein, the coating can be applied to at least one portion of a surface of a macrocarrier. In an embodiment, the coating is applied by spraying, dipping, spin coating, flow coating, physical vapor deposition, atomic layer deposition, ultraviolet enhanced deposition, evaporative deposition, nebulization, atomization, plasma deposition, or a combination thereof.

In an embodiment, the method disclosed herein further comprises a pre-application step wherein at least one portion of one surface of the macrocarrier is subjected to a pre-treatment step before the coating is applied. In an embodiment, a pre-treatment step prepares the surface before applying a coating. In an embodiment, the method comprises a pre-treatment step for cleaning, activating, drying, and/or etching at least a portion of a surface of a macrocarrier. In certain embodiments, such a pre-treatment step can be advantageous for improving the adhesion and/or the cross-linking of a coating to the macrocarrier.

In an embodiment, the method disclosed herein further comprise a post-application step wherein at least one portion of a coating on a macrocarrier is subjected to a further processing step. In an embodiment, a post-application step is advantageous for the aesthetics of the macrocarrier or to improve the quality and/or adhesion of the coated macrocarrier. In an embodiment, the post-application step is advantageous for transfer of biological material to a target cell. In an embodiment, the post-application step comprises drying the macrocarrier, sterilizing the macrocarrier, or a combination thereof.

In an embodiment, the method further comprises applying a microcarrier composition to at least one portion of a coated surface of a macrocarrier. In an embodiment, the microcarriers are suspended in a liquid. In an embodiment, the liquid comprises water, a solvent, an alcohol, or a combination thereof. In an embodiment, the liquid comprises ethanol. In an embodiment, the liquid comprises ethanol at greater than 50%, 55%, 60%, 70%, 80%, 90%, or 95% by volume or is 100% ethanol. In an embodiment, the microcarrier composition comprises nanoparticles wherein at least a portion of the nanoparticles are coated with a composition comprising one or more biological macromolecules. In an embodiment, the biological macromolecules comprise DNA, RNA, polynucleic acid, a protein, a ribonucleoprotein, genetic material, a gene-editing reagent, or combination thereof

In an embodiment, the microcarrier composition is applied at, near, or about the center of a surface of the coated macrocarrier. In an embodiment, a liquid microcarrier composition is applied via a pipette, syringe, and the like. In an embodiment, the method comprises drying the microcarrier composition such that the liquid is evaporated, and the microcarriers remain on the surface of the macrocarrier.

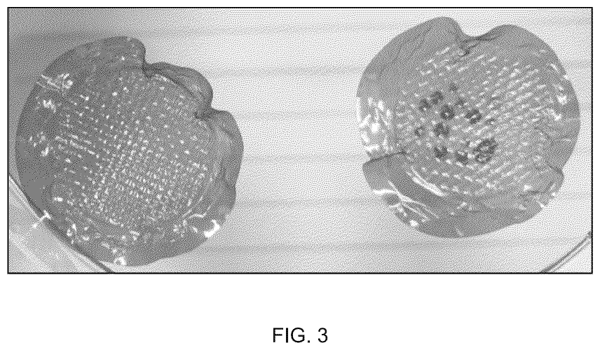

In embodiments in which the microcarrier composition is applied in an aqueous suspension, it is typically applied as several droplets near the center of the macrocarrier. While an aqueous suspension does not have tendency to spread over a dry macrocarrier similarly to an ethanol suspension, coated macrocarriers nevertheless have diminished microcarrier retention (similar to what is shown in FIG. 3).

Disclosed herein is a biolistic particle delivery system comprising a biolistic delivery apparatus comprising a gas acceleration tube, a rupture disk, a coated macrocarrier according to the disclosure herein, and a stopping screen. A non-limiting example of a biolistic delivery apparatus is shown in FIG. 1. In an embodiment, the coated macrocarrier further comprises a plurality of microcarriers as disclosed herein on at least a portion of a surface of the coated macrocarrier. In an embodiment, the microcarriers are coated with a composition comprising one or more biological macromolecules. In an embodiment, the biolistic particle delivery system further comprises target cells oriented in the system to receive at least a portion of the microcarriers coated with one or more biological macromolecules. Target cells can comprise plant cells, fungal cells, or animal cells.

Disclosed herein is a method of introducing one or more biological macromolecules into a cell. In an embodiment, the method comprises discharging a biolistic particle delivery system comprising a coated macrocarrier as described herein, thereby introducing one or more biological macromolecules into a cell. As used herein, the cell can comprise a plant cell, a fungal cell, or an animal cell. As used herein, “cell,” “target cell,” and “tissue,” and “target tissue” can be used interchangeably to indicate the targeted cell and/or tissue, a portion of which receives the particles in a biolistic particle delivery system.

In an embodiment, the method comprises discharging a biolistic particle delivery system comprising a coated macrocarrier introduces more biological macromolecules into a cell than a control biolistic particle delivery system that comprises a macrocarrier lacking the coating. In an embodiment, the increased introduction of biological macromolecules results in an increased rate of cell transformation when at least a portion of the biological macromolecules comprise a DNA molecule comprising a gene. In an embodiment, the increased introduction of biological macromolecules results in an increased delivery of gene-editing reagents when at least a portion of the biological macromolecules comprise one or more gene-editing reagents (e.g., Cas nucleases and gRNAs, TALENs, artificial zinc finger nucleases, and/or a donor DNA template).

In an embodiment, the transfer efficiency of microcarriers from a coated macrocarrier as disclosed herein is increased in comparison to the transfer efficiency of a control system wherein the microcarriers are delivered with a macrocarrier lacking the coating. In an embodiment, an increased transfer efficiency results in a reduction in retention of the microcarriers on a coated macrocarrier as described herein after discharging in comparison to retention of microcarriers on a macrocarrier lacking the coating.

In an embodiment, a coating on a macrocarrier does not impart significant toxicity to the cell via particle delivery.

All publications, patent applications, issued patents, and other documents referred to in this specification are indicative of the level of ordinary skill in the art to which this disclosure pertains and are herein incorporated by reference to the same extent as if each individual publication or patent application was specifically and individually indicated as incorporated by reference. Definitions that are contained in text incorporated by reference are excluded to the extent that they contradict definitions in this disclosure.

The present disclosure is further illustrated by the following examples, which should not be considered as limiting in any way.

### EXAMPLES

### Example 1. Description of Materials Used

Macrocarriers are from Bio-Rad, catalog number 1652335.

Treated macrocarriers were sprayed with store-bought, commercially available PTFE (Teflon).

Treated and untreated macrocarriers were then sterilized by rinsing the macrocarriers in 2-propanol and then autoclaving the macrocarriers.

Gold nanoparticles were prepared essentially as described by Banakar R., Wang K. (2020) Biolistic Transformation of Japonica Rice Varieties. In: Rustgi S., Luo H. (eds) Biolistic DNA Delivery in Plants. Methods in Molecular Biology, vol 2124. Humana, New York, NY. doi.org/10.1007/978-1-0716-0356-7_8, incorporated herein by reference in its entirety. The 0.6 μm gold nanoparticles from Bio-Rad, catalog number 1652262, were coated with a TransIT®-2020 transfection reagent solution having a fluorescent marker and selection marker-encoding plasmid, ribonucleoproteins (RNPs; a Cas protein bound with RNA), and a short DNA oligonucleotide for non-homologous end joining (NHEJ) insertion.

To prepare the macrocarriers with the gold nanoparticles, the coated gold nanoparticles were suspended in water or ethanol, applied to macrocarriers, and then dried in a hood for 20 to 30 minutes.

Bombardment was performed using a Biolistic® PDS-100/He Particle Delivery System from Bio-Rad, catalog numbers 165-2257 and 165-2250LEASE to 165-2255LEASE.

### Example 2

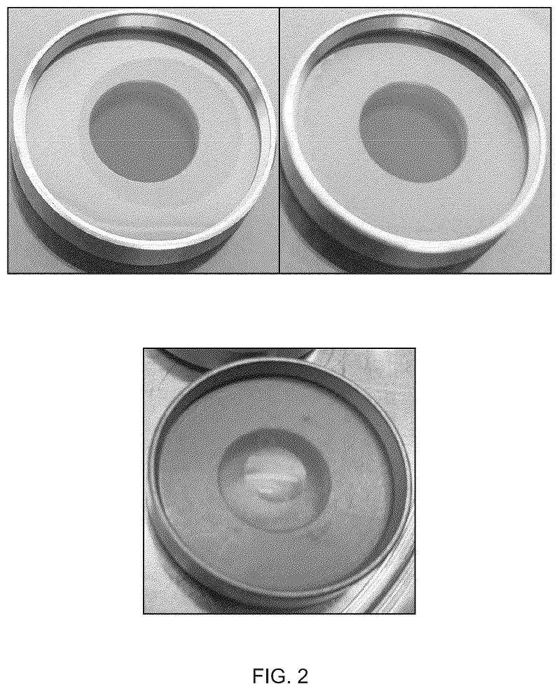

Materials described in Example 1 were used as follows in this Example. A sample of 100% ethanol which contained the gold nanoparticles was applied to the center of an untreated macrocarrier and a PTFE-treated macrocarrier. Photographs are shown in FIG. 2 wherein the untreated macrocarrier is at the top and the PTFE-treated macrocarrier is at the bottom.

As shown in FIG. 2, the ethanol spreads beyond the center of the untreated macrocarrier, outside the area of the stopping screen. For some macrocarriers, the spread is even greater, sometimes taking the gold nanoparticles away from the center making the macrocarrier unusable. In contrast, as shown in FIG. 2, the ethanol is contained within the center of the PTFE-treated macrocarrier. This indicates more of the gold nanoparticles will remain within the center of a macrocarrier with a hydrophobic coating efficiently transmitting the gold nanoparticles through the stopping screen. A sample of 100% ethanol which contained the gold nanoparticles was applied to the center of an untreated macrocarrier and a treated, PTFE-treated macrocarrier. Photographs are shown in FIG. 2 wherein the untreated macrocarrier is at the top and the treated macrocarrier is at the bottom.

### Example 3

Materials described in Example 1 were used as follows in this Example. Treated and untreated macrocarriers were used to deliver gold nanoparticles using a Bio-Rad Biolistic® PDS-100/He Particle Delivery System. Photographs of treated and untreated macrocarriers after bombardment are shown in FIG. 3, with the treated macrocarrier on the left and the untreated macrocarrier on the right. The uniform markings are from contact with the stopping screen.

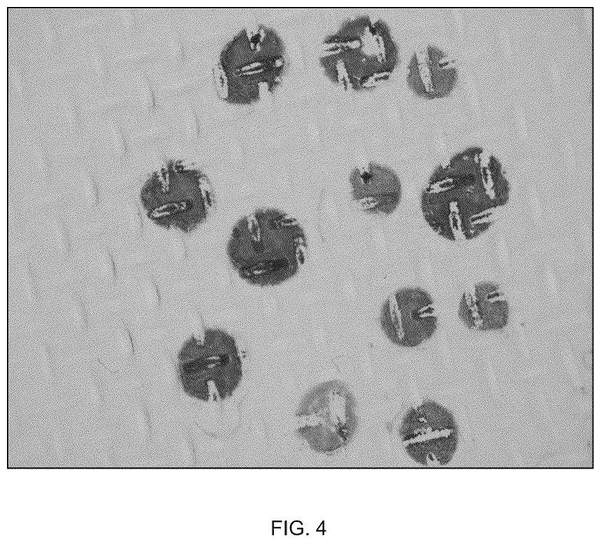

The treated macrocarrier as shown in FIG. 3 retains very few gold nanoparticles. In contrast, the untreated macrocarrier as shown in FIG. 3 retains some gold nanoparticles as indicated by the dark circular dots. FIG. 4 is a closeup photograph of an untreated macrocarrier after bombardment showing gold sticking to the macrocarrier. The PTFE-treated macrocarrier results in a significantly higher rate of passing the microcarrier load towards the target tissue.

### Example 4

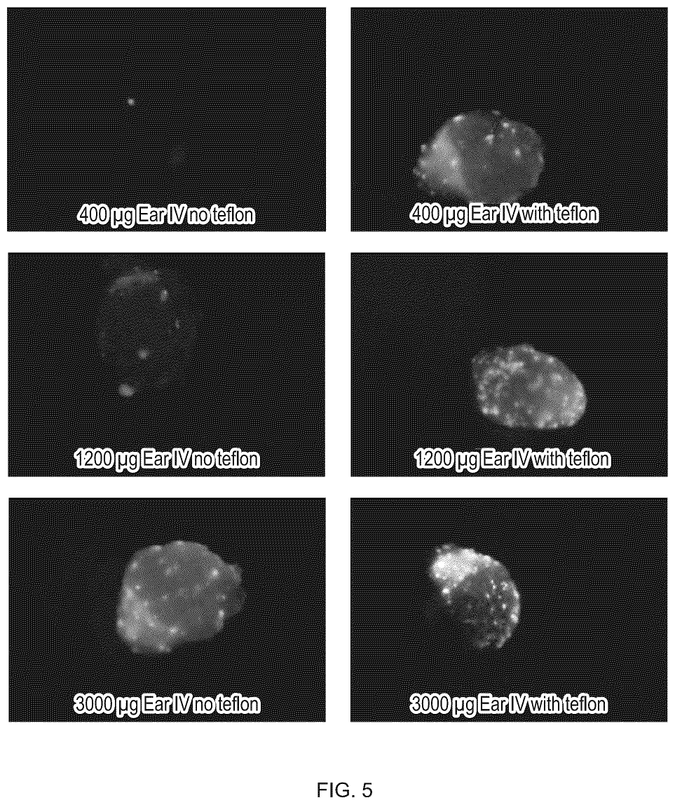

Materials described in Example 1 were used as follows in this Example. Immature embryos of the B104 maize variety were isolated 13 days after pollination. These embryos were bombarded with varying amounts of gold nanoparticles coated with the transfection reagent solution having the fluorescent and selection marker-encoding plasmid, RNPs, and the short DNA oligonucleotide for NHEJ insertion as described in Example 1. In the experiments, 400, 1200, or 3000 μg of the coated gold nanoparticles were applied to both the PTFE-treated surface of a test macrocarrier and to an untreated surface of a control macrocarrier. Four days post-bombardment the fluorescence of the embryos was observed by microscopy. Fluorescence images are shown in FIG. 5.

FIG. 5 (top panel at left) is an image of the embryo after bombardment with 400 μg gold nanoparticles utilizing an untreated macrocarrier and FIG. 5 (top panel at right) is an image of the embryo after bombardment with 400 μg gold nanoparticles utilizing a PTFE-treated macrocarrier. Comparing the two images, the PTFE-treated macrocarrier produced a significantly higher fluorescence than the untreated macrocarrier, indicating a higher transformation rate with the fluorescent marker gene.

FIG. 5 (middle panel at left) is an image of the embryo after bombardment with 1200 μg gold nanoparticles utilizing an untreated macrocarrier and FIG. 5 (middle panel at right) is an image of the embryo after bombardment with 1200 μg gold nanoparticles utilizing a treated macrocarrier. Similar to the 400 μg bombardment, the PTFE-treated macrocarrier produced a significantly higher fluorescence than the untreated macrocarrier, indicating a higher transformation rate with the fluorescent marker gene.

FIG. 5 (bottom panel) are fluorescence images of embryos after bombardment with an untreated (bottom panel at left) and a PTFE-treated macrocarrier (bottom panel at right) loaded with 3000 μg of coated gold nanoparticles. Both images show significant fluorescence. Too much microcarrier can result in tissue damage. It is considered ideal to use as little microcarrier as possible to accomplish the maximum viable transformation rates. The PTFE-treated macrocarriers enable more efficient biolistics transformation.

Three weeks after bombardment, calli from the embryos bombarded with coated microcarriers loaded on the PTFE-treated macrocarriers are indistinguishable from the embryos bombarded with coated microcarriers loaded on untreated macrocarriers. This indicates that the PTFE layer on the surface of the macrocarriers does not cause significant toxicity to the transformed tissue.

The present disclosure is further defined by the following numbered embodiments:
1. A coated macrocarrier comprising: a macrocarrier adapted for use in a biolistic particle delivery system; and a coating on a surface of the macrocarrier.
2. The coated macrocarrier of embodiment 1, wherein the coating comprises a hydrophobic material.
3. The coated macrocarrier of embodiment 2, wherein the coating comprises a hydrophobic polymer, optionally wherein the hydrophobic polymer is a fluoropolymer.
4. The coated macrocarrier of embodiments 2 or 3, wherein the coating comprises a fluoropolymer, optionally wherein the fluoropolymer comprises PTFE (polytetrafluoroethylene), PVDF (polyvinylidene fluoride), PCTFE (polychlorotrifluoroethylene), or a copolymer thereof.
5. The coated macrocarrier of embodiment 4, wherein the coating comprises PTFE.
6. The coated macrocarrier of embodiment 1, wherein the coating comprises a proteinaceous material.
7. The coated macrocarrier of embodiment 6, wherein the proteinaceous material comprises one or more proteins, glycoproteins, or a mixture thereof.
8. The coated macrocarrier of embodiment 6 or 7, wherein the proteinaceous material comprises a protein.
9. The coated macrocarrier of embodiment 8, wherein the protein comprises bovine serum albumin.
10. The coated macrocarrier of any of embodiments 1 to 9, further comprising a plurality of microcarriers on the surface of the macrocarrier having the coating, optionally wherein the microcarriers are nanoparticles comprising gold, tungsten, steel, or an alloy thereof.
11. The coated macrocarrier of embodiment 10, wherein the microcarriers are coated with a composition comprising one or more biological macromolecules.
12. The coated macrocarrier of embodiment 11, wherein the biological macromolecules comprise a polynucleic acid, a protein, a ribonucleoprotein, or combination thereof, optionally wherein the biological macromolecules comprising a gene-editing reagent or optionally wherein the gene-editing reagent comprises: (i) a Cas nuclease and/or a gRNA; (ii) a ribonucleoprotein comprising a Cas nuclease and a gRNA; (ii) a TALEN; (iii) an artificial zinc finger nuclease; and/or (iv) a donor DNA template.
13. The coated macrocarrier of any of embodiments 1 to 12, wherein the macrocarrier comprises a plastic.
14. A method of making a coated macrocarrier adapted for use in a biolistic particle delivery system comprising: applying a coating to at least one surface of a macrocarrier adapted for use in a biolistic particle delivery system which lacks the coating on a surface, thereby obtaining a coated macrocarrier adapted for use in a particle delivery system.
15. The method of embodiment 14, wherein the coating comprises a hydrophobic material.
16. The method of embodiment 15, wherein the coating comprises a hydrophobic polymer, optionally wherein the hydrophobic polymer is a fluoropolymer.
17. The method of any of embodiments 14 and 15, wherein the coating comprises a fluoropolymer, optionally wherein the fluoropolymer comprises PTFE (polytetrafluoroethylene), PVDF (polyvinylidene fluoride), PCTFE (polychlorotrifluoroethylene), or a copolymer thereof.
18. The method of embodiment 17, wherein the coating comprises PTFE.
19. The method of embodiment 14, wherein the coating comprises a proteinaceous material.
20. The method of embodiment 19, wherein the proteinaceous material comprises one or more proteins, glycoproteins, or a mixture thereof.
21. The method of embodiment 19 or 20, wherein the proteinaceous material comprises a protein.
22. The method of embodiment 21, wherein the protein comprises bovine serum albumin.
23. The method of any of embodiments 14 through 22, wherein the coating is applied to at least the one surface by spraying, dipping, spin coating, flow coating, plasma deposition, physical vapor deposition, atomic layer deposition, ultraviolet enhanced deposition, evaporative deposition, nebulization, atomization, or a combination thereof.
24. The method of any of embodiments 14 through 23, further comprising applying a microcarrier composition to the surface of the coated macrocarrier comprising the coating, optionally wherein the microcarrier composition is applied at, near, or about the center of the surface of the coated macrocarrier comprising the coating.
25. The method of embodiment 24, wherein the microcarrier composition comprises nanoparticles in a liquid.
26. The method of embodiment 25, wherein the liquid comprises ethanol, water, or a combination thereof, optionally wherein the liquid comprises ethanol at greater than 50%, 55%, 60%, 70%, 80%, or 90% by volume.
27. The method of any of embodiments 24 through 26, wherein the microcarrier composition comprises nanoparticles coated with one or more biological macromolecules.
28. The method of embodiment 27, wherein the biological macromolecules comprise a polynucleotide, a protein, a ribonucleoprotein, or combination thereof.
29. The method embodiment 25 or 26, further comprising drying the macrocarrier such that the liquid is evaporated and the nanoparticles remain on the surface of the macrocarrier.
30. A biolistic particle delivery system comprising: a biolistic delivery apparatus comprising a gas acceleration tube, a rupture disk, a coated macrocarrier according to any one of embodiments 1 to 13, and a stopping screen.
31. The biolistic particle delivery system of embodiment 30, wherein the macrocarrier further comprises a plurality of microcarriers on the surface of the macrocarrier having the coating and wherein the microcarriers are coated with a composition comprising one or more biological macromolecules.
32. The biolistic particle delivery system of embodiment 31, the system further comprising target cells oriented in the system to receive the microcarriers coated with one or more biological macromolecules.
33. A method of introducing one or more biological macromolecules into a cell, wherein the method comprises discharging the biolistic particle delivery system of embodiment 32, thereby introducing one or more biological macromolecules into a cell.
34. The method of embodiment 33, wherein the introducing of the one ore more biological macromolecules into the target cells with the macrocarrier comprising the coating is increased in comparison to a control system wherein one or more biological macromolecules are delivered with a macrocarrier lacking the coating.
35. The method of embodiment 34, wherein (i) the biological macromolecules comprise a DNA molecule comprising a gene and the increased introducing of the one or more biological macromolecules results in an increased rate of target cell transformation with the gene in comparison to the control system; and/or (ii) the biological macromolecules comprise one or more gene-editing reagents and the increased delivery of the gene-editing reagents results in an increased rate of gene-editing in comparison to the control system.

The above disclosure is intended to be illustrative and not exhaustive. The breadth and scope of the present disclosure should not be limited by any of the above-described embodiments.

What is claimed is:

1. A coated macrocarrier comprising:
a macrocarrier adapted for use in a biolistic particle delivery system; and
a coating on a surface of the macrocarrier.

2. The coated macrocarrier of claim 1, wherein the coating comprises a hydrophobic polymer or a proteinaceous material.

3. The coated macrocarrier of claim 2, wherein the coating comprises a fluoropolymer.

4. The coated macrocarrier of claim 3, wherein the fluoropolymer comprises PTFE (polytetrafluoroethylene), PVDF (polyvinylidene fluoride), PCTFE (polychlorotrifluoroethylene), or a copolymer thereof.

5-6. (canceled)

7. The coated macrocarrier of claim 1, wherein the coating comprises a proteinaceous material and wherein the proteinaceous material comprises one or more proteins, glycoproteins, or a mixture thereof.

8. (canceled)

9. The coated macrocarrier of claim 7, wherein the protein comprises bovine serum albumin.

10. The coated macrocarrier of claim 1, further comprising a plurality of microcarriers on the surface of the macrocarrier having the coating, wherein the microcarriers are coated with a composition comprising one or more biological macromolecules.

11. (canceled)

12. The coated macrocarrier of claim 10, wherein the biological macromolecules comprise a polynucleic acid, a protein, a ribonucleoprotein, or combination thereof.

13. (canceled)

14. A method of making a coated macrocarrier adapted for use in a biolistic particle delivery system comprising:
applying a coating to at least one surface of a macrocarrier adapted for use in a biolistic particle delivery system which lacks the coating on a surface, thereby obtaining a coated macrocarrier adapted for use in a particle delivery system,
wherein the coating comprises a hydrophobic polymer or a proteinaceous material.

15. (canceled)

16. The method of claim 14, wherein the coating comprises a fluoropolymer.

17. The method of claim 16, a wherein the fluoropolymer comprises PTFE (polytetrafluoroethylene), PVDF (polyvinylidene fluoride), PCTFE (polychlorotrifluoroethylene), or a copolymer thereof.

18-19. (canceled)

20. The method of claim 14, wherein the proteinaceous material comprises one or more proteins, glycoproteins, or a mixture thereof.

21. (canceled)

22. The method of claim 24, wherein the proteinaceous material comprises a protein, and wherein the protein comprises bovine serum albumin.

23. The method of claim 14, wherein the coating is applied to at least the one surface by spraying, dipping, spin coating, flow coating, physical vapor deposition, atomic layer deposition, ultraviolet enhanced deposition, evaporative deposition, nebulization, atomization, plasma deposition, or a combination thereof.

24. The method of claim 14, further comprising applying a microcarrier composition to the surface of the coated macrocarrier comprising the coating, wherein the microcarrier composition is applied at, near, or about the center of the surface of the coated macrocarrier comprising the coating.

25. The method of claim 24, wherein the microcarrier composition comprises nanoparticles in a liquid.

26. The method of claim 25, wherein the liquid comprises ethanol, water, or a combination thereof, optionally wherein the liquid comprises ethanol at greater than 50%, 55%, 60%, 70%, 80%, or 90% by volume.

27. The method of claim 24, wherein the microcarrier composition comprises nanoparticles coated with one or more biological macromolecules, wherein the biological macromolecules comprise a polynucleotide, a protein, a ribonucleoprotein, or combination thereof.

28. (canceled)

29. The method of claim 25, further comprising drying the macrocarrier such that the liquid is evaporated and the nanoparticles remain on the surface of the macrocarrier.

30. A biolistic particle delivery system comprising:
a biolistic delivery apparatus comprising a gas acceleration tube, a rupture disk, a coated macrocarrier according to claim 1, and a stopping screen.

31-32. (canceled)

33. A method of introducing one or more biological macromolecules into a cell, wherein the method comprises discharging the biolistic particle delivery system of claim 30, thereby introducing one or more biological macromolecules into a cell.

34-37. (canceled)
